(19) United States
(12) Patent Application Publication
(10) Pub. No.: US 2010/0093849 A1
(43) Pub. Date: Apr. 15, 2010

(54) OXALIPLATIN PHARMACEUTICAL COMPOSITION WITH ALCOHOLIC SUGAR-BASED BUFFER

(76) Inventors: Borek Zaludek, Brno (CZ); Libuse Zatloukalová, Brno (CZ); Frantisek Zák, Brno (CZ); Bozena Matejková, Brno (CZ); Hana Tkadlecková, Brno (CZ)

Correspondence Address: NOTARO & MICHALOS P.C., 100 DUTCH HILL ROAD, SUITE 110, ORANGEBURG, NY, 10962-2100, US

(21) Appl. No.: 12/523,098

(22) PCT Filed: Jan. 10, 2008
(86) PCT No.: PCT/CZ2008/000006

§ 371 Date: Nov. 3, 2009

(30) Foreign Application Priority Data
Jan. 22, 2007 (CZ) PV 2007-48
Jan. 22, 2007 (CZ) PV 2007-49

(51) Int. Cl. A61K 31/282

(52) U.S. Cl. 514/492

(57) ABSTRACT

A pharmaceutical composition, intended particularly for potential administration and for treatment of tumor diseases sensitive to oxaliplatinum, comprises oxaliplatinum as the active compound, a pharmaceutically acceptable aqueous solvent and a stabilizing agent in stabilizingly effective amount. The stabilizing agent includes at least one compound selected from the group consisting of acids derived from neutral alcoholic sugars, lactones of these acids and salts of these acids. In a method of producing such a pharmaceutical composition, oxaliplatinum is dissolved in an aqueous solvent, whereupon to the obtained oxaliplatinum solution is added at least one acid derived from a neutral alcoholic sugar and/or at least one lactone of these acids and/or at least one salt of these acids, and optionally the pH value of the solution is adjusted by addition of an alkali metal hydroxide and/or an alkali earth metal hydroxide to pH 3.5-6.5, whereupon the obtained solution is sterilized and filled into individual package units and optionally inertized with nitrogen or argon.

### BACKGROUND OF THE INVENTION

Oxaliplatinum is an antineoplastic compound belonging to the group of platinum derivatives in which the platinum atom is bonded in a complex with 1,2-cyclohexanediamine and an oxalate ligand. In its optically pure form, oxaliplatinum was first prepared in 1978 by Kidani by isolation from a mixture of isomers. Chemically it is {trans-(1R,2R)-1,2-cyclohexane-diamine}-(oxalato)platinum(II) complex.

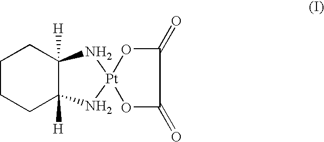

Oxaliplatinum (I) is a white, finely crystalline substance of limited solubility in water (about 8 mg/ml at 37° C.), practically insoluble in ether and ethanol.

Oxaliplatinum exhibits a broad spectrum of in vitro cytotoxicity as well as in vivo antitumor activity in a wide spectrum of tumor model systems. It is also active in vitro as well as in vivo in cisplatinum-resistant models. It has been found that in combination with 5-FU oxaliplatinum exhibits a synergistic cytotoxic effect in vitro as well as in vivo.

The drug form of oxaliplatinum which is hitherto worldwide most sold is the preparation manufactured by lyophilization of a solution of oxaliplatinum and a suitable constituting substance.

Prior to application to a patient, it is necessary to reconstitute the lyophilizate and to dilute the obtained solution with an infusion medium—5% glucose solution—to final concentration 0.2-0.7 mg/ml. Oxaliplatinum is administered in the form of infusion into a peripheral vein, or using a central vein catheter, usually in a dose of 85 mg/m², for 2 to 6 hours.

Oxaliplatinum is synthesized in several steps. The fundamental synthetic way is described in the work of Kidani (Kidani Y. at al, Gann, Vol. 71, 637; Chem. Abstr. Vol. 94, 4129d), in Japanese patent application JP 53031648 A (Chem. Abstr. Vol. 89, 199862y) and in American patent U.S. Pat. No. 4,169,846. The first step of the synthesis comprises reaction of potassium tetrachloroplatinate(II) with trans-(1R,2R)-isomer of 1,2-cyclohexanediamine. In this reaction two of the chlorine ligands in the tetrachloroplatinate(II) anion are replaced by the amino groups of the bivalent ligand under formation of complex [PtCl₂(dach)] with closure of a thermodynamically stable five-membered ring involving the central platinum atom of favorable steric arrangement. In the next step, reaction with silver nitrate affords aqua complex [Pt(dach)(H₂O)₂]²⁺ which is treated with oxalic acid or potassium oxalate. This reaction gives oxaliplatinum the bivalent oxalate ligand of which again forms a stable five-membered ring with the central platinum atom.

This generally employed synthetic route, described in the literature, or its modifications according to a whole series of inventions, suffers from a drawback consisting in use of great number of organic as well as inorganic agents which, as accompanying impurities, may contaminate the final product, i.e. oxaliplatinum.

Particularly, such impurities may be alkali metal ions (sodium or potassium ions and alkali earths metal ions), silver ions, and the corresponding anions, i.e. chloride, iodide, nitrate or acetate ions and, last but not least, also residual oxalate ions. The mentioned accompanying impurities then reduce the quality of the drug form employed for an infusion preparation.

As generally known, in aqueous solutions oxaliplatinum decomposes under formation of monomeric and dimeric aqua complexes in which the central platinum atom has oxidation number II. Degradation products with central platinum atom of oxidation number IV may also be formed. The amount of these degradation products then significantly influences the toxicological profile of the preparation. With regard to the current use of oxaliplatinum in the medical practice—in the form of intravenous infusion—as described above, it is mandatory to assure a high and long-lasting guaranteed quality, and thus also safety, of this drug form.

One of the possibilities how to ensure a long-lasting quality of the preparation is to use lyophilization of a suitable composition; in this process a solution of the composition is first freezed and then the content of the solvent—usually water—is reduced by sublimation at diminished temperature and then by resorption to a level preventing degradative chemical reactions.

From the viewpoint of preparation of the final application form—a liquid infusion solution—the lyophilizate suffers from certain disadvantages. It has to be first reconstituted and only then diluted to the desired concentration. During the reconstitution, in some cases it is necessary to shake the solution in order to dissolve completely the lyophilization cake. This lengthy procedure increases the risk of contamination of workers and environment, which is undesirable because of toxicity of the compound. At the same time, the procedure increases the possibility of microbial contamination of the product itself.

Another evident disadvantage is that production of the lyophilized form is technologically and economically demanding because pharmaceutical production of lyophilized forms is very expensive and requires special equipment and skilled workers.

Disadvantages connected with complicated preparation of the final application form by reconstitution and dilution of the lyophilizate and with economically demanding production of the lyophilized form are solved by a liquid form of the oxaliplatinum-containing preparation which, however, must be of sufficient stability and therapeutic effectivity, comparable with the lyophilized form.

This objective has been pursued by many authors who to this end tried various approaches. One of them was to introduce more or less effective purifying processes in the course of the synthesis of the active substance—oxaliplatinum—and to prepare a product free of accompanying impurities that might catalyze its decomposition. Thus, according to EP 0617043, residual silver ions, remaining in oxaliplatinum solutions after removal of silver chloride by filtration, were precipitated by addition of sodium or potassium iodide.

The authors of JP 5301884 purified the prepared aqua complex by reverse osmosis and obtained thus a final product that contained markedly less accompanying impurities such as the anions and cations mentioned.

EP 567438 describes a method that makes use of high pressure liquid chromatography for the preparation of high purity oxaliplatinum.

Effective purification of the active compound increases costs of its preparation, and thus also the price of the final drug form, as the result of losses in individual classical purification procedures and contingently also due to expenses combined with the suggested approaches based on modern separation techniques.

Preparation of oxaliplatinum of high purity made it possible to obtain a liquid drug form comprising a solution that contains only the active compound and water, as described in patent application WO9604904 which discloses a oxaliplatinum-based pharmaceutically stable preparation for instant parenteral application, which is an aqueous solution of oxaliplatinum in concentration 1-5 mg/ml, has pH in the range 4.5-6.5, and which on storing for an acceptable time remains clear, colorless and without precipitate, and in which the content of the active compound does not drop below 95% of the original value.

However, decomposition of the active compound may be catalyzed even by trace amounts of impurities that cannot be detected by usual checking methods and may be present even in the purified active compound.

Therefore, aqueous solutions of oxaliplatinum were not always sufficiently stable even if they had been prepared from purified substance. Further studies were therefore aimed at attempts to increase the stability of a liquid oxaliplatinum-containing preparation by addition of various stabilizers that should protect the active compound from negative influences of accompanying impurities initiating its decomposition.

WO9943355 discloses a pharmaceutically stable composition formed by a solution of oxaliplatinum, its use in the therapy of carcinomas and its preparation, including a method of stabilization of oxaliplatinum solution. This method comprises addition of an effective stabilizing amount of a buffer, based on oxalic acid or its alkali metal salt, to an aqueous solution of oxaliplatinum. The stabilization is based on the Le Chatelier's principle of reaction equilibrium shift. Since decomposition of oxaliplatinum gives rise to oxalic acid, it is assumed that, in an equilibrium system, its addition may suppress the decomposition. However, oxalic acid is toxic and can damage kidneys and other organs. The suitability of stabilization of oxaliplatinum solutions by oxalic acid and/or its salts is questionable also because it does not consider the fact that this is the case of a chelate bond of ligands in a complex. The unsuitability of this stabilization method is, inter alia, illustrated by the fact that other patent application (WO 03004505) relates to preparation of oxaliplatinum that has been further purified to remove oxalic acid.

Another method of stabilization of an oxaliplatinum-based preparation is described in WO03047587. The stabilization of the liquid formulation practically consists in addition of an effective amount of lactic acid, its salts or a lactate buffer, as a stabilizing agent, into an aqueous solution of oxaliplatinum.

US2003109515 discloses a stable liquid injection form containing malonic acid and/or its salt and a pharmaceutically acceptable vehicle, defined as water. The claims further specify the concentration of malonic acid or its salt, pH of the solution, the amount of oxaliplatinum (up to 10 mg/ml), the purity and melting point of oxaliplatinum, the method of producing injection solution and its use.

WO0115691 discloses a liquid preparation, containing oxaliplatinum in concentration of at least 7 mg/ml in a solvent containing a sufficient amount of at least one hydroxy derivative selected from the group comprising 1,2-propanediol, glycerol, maltitol, sucrose and inositol. In this case, however, the patent concerns not the stabilization of injection preparations but rather increase of solubility of oxaliplatinum. The “sufficient amount” of solubilizers in the solvent is at least 10%, the highest concentrations of oxaliplatinum (14.01 or 14.33 mg/ml) were achieved in a solvent consisting of the same volumes of 1,2-propanediol and water.

Although WO0115691 as such does not concern the stability of the preparations, the related WO 2002047725 concerns thermally sterilized liquid injection forms of oxaliplatinum of concentration at least 7 mg/ml which contain the same solubilizers and are so stable that they withstand heating to high temperatures.

In addition to the compounds claimed in WO0115691, also some other polyhydroxy compounds have been examined, such as lactose, mannitol, sorbitol, polyalkylene glycols and cyclodextrins. According to the data in the mentioned patent application, these compounds, however, did not work well. Some other oligosaccharides tested were reported to be good solubilizers, however, they are too expensive. WO0115691 also relates to various packaging types of the preparation suitable for parenteral administration, including infusion bags and pre-filled syringes, as well as a method of preparing a liquid drug form.

Patent application EP1466599 relates to a pharmaceutical composition comprising water, oxaliplatinum and a physiologically acceptable sugar, further specified as glucose, fructose, galactose, sucrose, maltose, trehalose and dextran, or their mixtures. The sugar concentration is at least 5%, oxaliplatinum concentration should be 5-25 mg/ml. In addition to sugars, the liquid composition may further contain tonifying (osmotic pressure adjusting) substances or pH adjusting substances, or preservatives. The solutions may contain acids, further specified as phosphoric, sulfuric, methanesulfonic, ethanesulfonic and p-toluenesulfonic acid and mixtures thereof.

Patent application EP1466600 discloses a composition comprising oxaliplatinum, water and an inorganic or organic acid, excluding oxalic acid, “the anions of which do not influence stability of the solution”. The acid is then specified as phosphoric, sulfuric, methanesulfonic, ethanesulfonic and p-toluenesulfonic acid and mixtures thereof. Further the claims concern the use of one or more inorganic or organic acids (excluding oxalic acid) for the preparation of a stable aqueous solution of oxaliplatinum, and the use of acids for stabilization of oxaliplatinum solutions.

According to claim 3 of the said patent application, the composition may further contain excipients regulating osmotic pressure or pH, or unspecified preservatives. Claim 6 gives a wide margin of oxaliplatinum concentration, from 0.025-25 mg/ml.

Patent application US20050090544 concerns the stabilization of an oxaliplatinum-containing composition intended for parenteral administration, using tartaric acid, salts and derivatives thereof. The equivalent application WO2005020980 claims addition of in principle any “stabilizing acid”, excluding oxalic, lactic and malonic acids, to a liquid pharmaceutical formulation with oxaliplatinum intended for parenteral administration. In the second claim of WO2005020980, the stabilizing acids are specified as carboxylic acids, in the third claim as dicarboxylic acids, and then citric, maleic, saccharic (glucaric), succinic, malic, and tartaric acids, and mixtures thereof. Finally, the selection is reduced to tartaric acid. However, some of the acids listed in the claims are evidently not usable as stabilizers, which is documented by the examples described directly in WO2005020980.

Stability tests have shown that after storing at 40° C. for 5 weeks, a composition with added lactic acid deposited small amount of black particles, apparently reduced metallic platinum, whereas in a composition, prepared using saccharic acid, greater amount of white particles were found (see Table 22, p. 33 in WO2005020980). The insufficient stabilizing effect of several other acids, listed in the claims of WO2005020980, follows from other documents. The use of succinic acid has been described already earlier in Examples in patent application US20030109515, even with reference to U.S. Pat. No. 6,306,902, but it has not been claimed in any of the documents. Use of acidic citrate buffer, pH 3, formed by citric acid and its salt, has also been described in a patent document, but again it has not appeared in any of the claims (see the above-cited WO9943355, p. 16, Table 2, and its American equivalent U.S. Pat. No. 6,306,902).

WO2005102312 protects a liquid pharmaceutical formulation for parenteral administration comprising more than 5 mg/ml of oxaliplatinum, water and an effective amount of cyclodextrin as solubilizer. The invention further relates to use of the formulation in treatment of cancer, and to a method of producing this pharmaceutical formulation.

According to patent application WO 2006/048194, stabilizing effect on a liquid drug form of oxaliplatinum for parenteral administration was observed on addition of sodium acetate or an acetate buffer which, however, must be present in only very small concentration—0.005 to 0.00005 M. The best stabilization results were obtained at pH 4-6.

The concentration of oxaliplatinum in a stabilized solution may range between 0.1 and 10 mg/ml, preferably between 2 and 5 mg/ml.

As shown above, attempts to stabilize and/or solubilize oxaliplatinum in water have been made using many auxiliary substances, organic or inorganic acids, mono-, di- and oligosaccharides or polymeric compounds, sometimes with surprisingly contradictory chemical properties. These studies were done with the aim to primarily stabilize the effective compound—oxaliplatinum, irrespective of negative effect of accompanying impurities. Apparently, the stabilizing compounds were selected by chance, without understanding the causes of decomposition of the active compound. This is also the reason why the results described in the cited patent applications are contradictory. A compound claimed in one document as a potent stabilizer, according to other authors showed no stabilizing activity. The inconsistency and nonreproducibility of results can be explained by different content of catalytically effective impurities in the active compound. Such impurities are generally present in trace amounts that are not detectable by currently used analytical procedures and thus cannot be found in quality checks of oxaliplatinum.

Liquid compositions of oxaliplatinum, consisting in solution of the active compound in an aqueous vehicle, are only seemingly simple. In actual fact, they represent a very complex system the components of which interact with each other, their interactions influencing the behavior and properties of the system. The decomposition of oxaliplatinum in aqueous systems is influenced by a number of physical as well as chemical factors and gives rise to various products that are either inactive or, in a worse case, have serious adverse effects.

An important decomposition reaction is the loss of the oxalate ligand and its replacement with water under formation of aqua complexes. If the solution contains some substances capable of forming platinum complexes, then the oxalate moiety, or contingently water molecules, in the primarily arising aqua complex may be replaced also by other ligands, e.g. carboxylate ligands, in case that the solution contains carboxylic acids and/or their salts.

Other decomposition reactions are connected with change in oxidation state of platinum in the complex—either oxidation to a tetravalent platinum complex or, on the contrary, reduction of the complex to metallic platinum which then may deposit in the form of black particles.

Although in solutions of oxaliplatinum in pure water such decomposition reactions are relatively slow, their rate is not negligible and may be significantly enhanced by the presence of catalytically active impurities.

The loss of the oxalate ligand is a hydrolytic reaction which generally may be catalyzed by acids or bases.

In an aqueous medium, acids or bases are entirely or partly ionized under formation of a conjugated acid-base pair. Also water itself is to a certain extent ionized, forming a conjugated pair formed by the hydronium cation and the hydroxyl anion. Other water molecules are bound by hydrogen bonds to both the ions, giving thus rise to hydrated hydronium or hydroxyl ions such as H₇O₃⁺ and H₉O₄⁺ or H₇O₄⁻ which then take part in hydrolytic reactions. If the solution contains other compounds such as carboxylic acids, still more complex equilibria arise in the system in which, in addition to hydrated hydronium and hydroxyl ions, pairs of the carboxylic acid, R—COOH, and its conjugated base, R—COO⁻, also participate. The catalytic effect of acids and bases depends on their concentration and their strength. In equilibrium systems the concentration of acids and their conjugated bases depends on pH of the medium. The measure of acid strength is the value of dissociation constant, pKa. The more readily an acid dissociates, the stronger it is and, conversely, the weaker is its conjugated base.

It is thus evident that by adjusting pH by addition of acids or of buffers formed by a mixture of the acid and its salt it is possible to influence also the effectivity of acid-base catalysis in the decomposition of aqueous solutions of oxaliplatinum. In theory, aqueous solutions of oxaliplatinum should be most stable at neutral value of pH.

The cleavage of oxalic acid from the oxaliplatinum molecule is a two-step equilibrium process. In the first step, the oxalate five-membered ring is cleaved under formation of a monodentate oxalate intermediate which, in the second step, loses an oxalate ion under formation of diaqua complex. The rates of both the reactions depend on the concentration of hydroxyl ions, the opening of the oxalate ring being about 6 times faster than the loss of the oxalate ion (E. Jerrelmam et al., J. Pharm. Sci, 2002, 91(10): 2116-2121). Because of this equilibrium reaction, the optimum stability of aqueous solutions of oxyliplatinum is shifted to weakly acidic region, i.e. to pH 4-6. If the acidity of oxaliplatinum solutions is still higher, then the stability of the platinum complex decreases as the result of the acid-catalysed decomposition.

Bases present in the solution may replace water in the arising aqua complexes and form various carboxylate complexes. The stronger the base, the higher the probability of formation of such complex. Oxalic acid is a relatively strong dicarboxylic acid; its first and second dissociation constant, pKₐ, is 1.27 and 4.28, respectively (Merck Index). Therefore, the corresponding monoanion is a weak base and the basicity of the dianion is comparable with, or lower than, basicity of anions of organic acids used in the preparation of current buffers.

If the decomposition of oxaliplatinum liberates an oxalate anion and the solution contains an anion of other organic acid, which anion is a stronger base, the equilibrium may be disturbed so that in the reverse reaction another carboxylate complex may arise instead of the monodentate oxalate complex. If the acid molecule contains a further group that can bind to the central platinum atom, another bidentate cyclic complex may arise which is more stable than the acyclic complexes. Naturally, the course of the competing reactions depends also on the base concentration which in an equilibrium system depends on the amount of the components added as well as on pH of the solution that determines the dissociation degree.

Beside the acid-base catalysis, also an undesired catalytic effect of metal ions present may operate in the decomposition of oxaliplatinum. These metal ions usually do not affect the active components directly but they catalyze the formation of free radicals which then attack the active compound. Thus, e.g., in an aqueous medium, the presence of ferric ions may give rise to hydroxyl or superoxide radicals which then oxidize various compounds. Oxidation of oxaliplatinum gives rise to a tetravalent platinum complex of entirely different properties, thus decreasing the concentration of the active compound in the composition.

The presence of trace concentrations of iron in aqueous pharmaceutical compositions cannot be excluded because most of the equipment and tubing for distribution of water for injections is made of steel.

In liquid compositions of various drugs intended for parenteral administration, metallic impurities are usually masked by addition of chelating compounds of the EDTA type. However, in the case of drugs such as oxaliplatinum, which in itself has a metal complex character, one has to take into account also the possibility of displacement of the original ligands and their replacement by ligand of the chelating agent added. This then results in a lower amount of the active component and thus lower therapeutic effect. Very strong complexes of EDTA with bivalent platinum are known and studied since the fifties of the last century. To suppress the undesired catalytic effect of metal ions on the stability of platinum cytostatics it is therefore necessary to use compounds that can mask the undesired ions in solution but do not decompose the therapeutically active platinum complexes.

During analysis of behavior of oxaliplatinum in aqueous solutions and study of negative effects of accompanying impurities and additives on composition stability, the authors of the present invention arrived at significant original findings which made it possible to suggest a method of stabilization of a liquid composition containing oxaliplatinum as active component, and thus in this way to prepare stable sterile drug forms of a substance suitable for parenteral application, without necessity to purify the substance used by special separation techniques.

### SUMMARY OF THE INVENTION

The present invention relates to a pharmaceutical composition that comprises oxaliplatinum and an alcoholic sugar-based stabilizer, and to a method of producing such a pharmaceutical composition.

In one embodiment, the present invention is directed to a pharmaceutical composition comprising oxaliplatinum, a pharmaceutically acceptable aqueous solvent and a stabilizing agent in a stabilizingly effective amount. The stabilizing agent includes at least one compound selected from the group consisting of acids derived from neutral alcoholic sugars, lactones of these acids, and salts of these acids.

In another embodiment, the present invention is directed to a method of producing a pharmaceutical composition. In one specific embodiment of the method, oxaliplatinum is dissolved in an aqueous solvent to form an aqueous oxaliplatinum solution. The aqueous oxaliplatinum solution is combined with a stabilizing agent that includes at least one compound selected from the group consisting of acids derived from neutral alcoholic sugars, lactones of these acids, and salts of these acids to form an oxaliplatinum-stabilizer mixture. Optionally, the pH of the oxaliplatinum-stabilizer mixture is adjusted to a pH value between 3.5 and 6.5 by addition of an alkali metal hydroxide and/or an alkali earth metal hydroxide to the oxaliplatinum-stabilizer mixture to there by form a stable oxaliplatinum solution. The stable oxaliplatinum solution is sterilized. The sterilized, stable oxaliplatinum solution is filled into individual package units and optionally inertized with nitrogen or argon.

In another specific embodiment of the method of the invention, the pharmaceutical composition is prepared by mixing oxaliplatinum and a stabilizing agent that includes a lactone of an acid derived from a neutral alcoholic sugar (e.g., preferably an equilibrium aqueous mixture of a lactone of an acid derived from a neutral alcoholic sugar, and its corresponding acid) to form an oxaliplatinum-stabilizer mixture. The pH of the oxaliplatinum-stabilizer mixture is optionally adjusted to a pH value between 3.5 and 6.5 by addition of an alkali metal hydroxide and/or an alkali earth metal hydroxide to the oxaliplatinum-stabilizer mixture to form a stabilized oxaliplatinum solution.

The pharmaceutical compositions of the invention have substantially high stability in an aqueous medium (see Examples A and B). Thus, the pharmaceutical compositions of the invention can be used for parenteral administration to a subject, for example, to treat oxaliplatinum-sensitive tumor diseases.

### DETAILED DESCRIPTION OF THE INVENTION

The invention relates to a pharmaceutical composition, such as a sterile liquid pharmaceutical composition, comprising oxaliplatinum as active component, a pharmaceutically acceptable aqueous solvent and a stabilizing ingredient in stabilizingly effective amount, wherein the stabilizing agent includes at least one compound selected from the group consisting of acids, preferably monocarboxylic acids, derived from neutral alcoholic sugars, lactones of these acids and salts of these acids.

Typically, the concentration of oxaliplatinum is in an amount of between 1 mg/ml and 10 mg/ml. In a specific embodiment, the concentration of oxaliplatinum is in an amount of between 3 mg/ml and 7 mg/ml of the composition. In another specific embodiment, the concentration of oxaliplatinum is in an amount of between 3 mg/ml and 6 mg/ml of the composition. In yet another specific embodiment, the concentration of oxaliplatinum is in an amount of 5 mg/ml.

Typically, the concentration of at least one acid derived from neutral alcoholic sugars and/or at least one lactone of this acid and/or at least one salt of this acid, in total, is in a range of between 0.0005 mg/ml and 0.5 mg/ml of the composition. In one specific embodiment, the concentration in total is in a range of between 0.005 mg/ml and 0.1 mg/ml. In another specific embodiment, the concentration in total is in a range of between 0.01 mg/ml and 0.05 mg/ml. In yet another specific embodiment, the concentration in total is in a range of between 0.015 mg/ml and 0.025 mg/ml, such as in an amount of 0.018 mg/ml.

In a specific embodiment, as the stabilizing agent the composition according to the invention includes at least one acid, more preferably a monocarboxylic acid, derived from mannitol and/or sorbitol and/or lactones of this acid and/or salts of this acid. In another specific embodiment, as the stabilizing agent the composition includes an acid, more preferably a monocarboxylic acid, derived from neutral alcoholic sugars, which in aqueous solution is at equilibrium with its lactone. In yet another specific embodiment, as the stabilizing agent the composition includes gluconic acid and/or gulonic acid and/or mannonic acid and/or lactones and/or salts thereof. In yet another specific embodiment, as salts of acids derived from neutral alcoholic sugars the composition includes alkali metal salts and/or alkali earth metal salts of these acids, specifically sodium and/or potassium and/or magnesium and/or calcium salts of the said acids.

Studies on stabilization of aqueous solutions of oxaliplatinum within the framework of the invention show that, as stabilizing compounds in the invention, carboxylic acids, particularly monocarboxylic acids derived from the structure of neutral alcoholic sugars such as e.g. sorbitol or mannitol, i.e. gluconic acid or gulonic acid or mannonic acid and their lactones as well as their salts, can provide stable aqueous solutions of oxaliplatinum. Although the invention will be described in detail using the said three acids, the facts mentioned below relate also to other defined acids.

The mentioned acids are advantageous because they exist in the form of lactones which in an aqueous medium are only partly ionized, and in solution they form an equilibrium mixture of lactone and acid. Thus, e.g. gluconic acid may be added to the mixture in the form of glucono-δ-lactone and in the solution an equilibrium between δ-lactone, γ-lactone and the free acid is established. The equilibrium is established rapidly, as follows from optical rotation measurements or studies of spectroscopic changes or changes of pH of the solution (Y. Pocker, E. Green, Hydrolysis of D-glucono-δ-lactone. I. General Acid-Base Catalysis, Solvent Deuterium Isotope Effects, and Transition State Characterization, J. Am. Chem. Soc. 1973, 95(1): 113-119). Glucono-δ-lactone is a defined crystalline substance which crystallizes on concentration of gluconic acid solutions. On the other hand, gluconic acid is currently commercially available in the form of its 50% aqueous solution and preparation of the crystalline acid requires specific procedures. For use in drug forms, preparation of a gluconic acid solution by dissolving its δ-lactone is advantageous because glucono-δ-lactone and methods of its analysis are described in the pharmacopoeia whereas gluconic acid itself not. Herein after, the term “gluconic acid” will be used for a solution obtained by dissolution of glucono-δ-lactone in water.

The hydrolysis of oxaliplatinum can be suppressed by lowering the pH value of the composition. This may be achieved by use of various acids and/or buffers but, as evident from the above-mentioned patent applications, the choice of suitable agent cannot be generalized, because many acids and/or their salts are reported to influence negatively the stability of oxaliplatinum in solution. However, according to the present invention, the stabilization can be achieved with D-gluconic acid or a mixture of D-gluconic acid and a salt thereof. Gluconic acid and/or salts thereof exhibit a number of properties that are advantageous for stabilization of oxaliplatinum. Gluconic acid is a polyhydroxy compound which may, similarly to mannitol or sorbitol, to a certain extent stabilize the structure of water and thus decrease the availability of water molecules as reaction components in hydrolysis of the platinum complex. It is a monocarboxylic acid of medium strength, of dissociation constant 3.70, and therefore its conjugated base is not very strongly basic. It can form platinum complexes, and such complexes were prepared even as an antitumor medicament (Kidani et al., U.S. Pat. No. 4,477,387) of advantageous properties, however, the preparation requires a prior conversion into the diaqua complex, is considerably time-consuming, and requires the use of at least equimolar amount of sodium gluconate. The reaction does not proceed completely and the reaction mixture includes, in addition to the desired digluconato complex, significant amounts of the starting diaqua complex and the intermediary monoaqua-monogluconato complex.

Studies on stabilization of liquid pharmaceutical oxaliplatinum compositions revealed that addition of a small amount of gluconic acid or mixtures of gluconic acid and its salt to the composition can lead to adjustment and stabilization of pH value, so that hydrolysis of oxaliplatinum to the diaqua complex is suppressed but the gluconate concentration in the solution is not sufficient enough to enable an effective bonding of the gluconate ligand.

The use of gluconic acid has still another important effect on stability of the composition. Whereas gluconate complexes of platinum arise in oxaliplatinum solutions only with difficulty, gluconic acid can easily form stable complexes with metal ions that may be present in trace amounts in the composition. This concerns particularly complexes of trivalent iron ions, but also ions of nickel, cobalt, chromium, copper etc. (D. T. Sawyer, Metal-Gluconate Complexes, Chem. Rev. 1964, 64, 633-643) which may contaminate the solution from metallic materials used in the manufacturing.

Like D-gluconic acid, also mannonic or gulonic acids affect similarly the stability of aqueous oxaliplatinum compositions. However, neither these acids nor their lactones are hitherto listed among pharmacopoeial excipients which complicates their utilization in producing a pharmaceutical formulation.

In a specific embodiment, a gluconic, gulonic or mannonic acid is employed, and the concentration of gluconic or gulonic or mannonic acids may range between 0.0005 mg/ml and 0.5 mg/ml of the composition. Alternatively, the concentration of the gluconic, gulonic or mannonic acid is in a range of between 0.005 mg/ml and 0.1 mg/ml, such as between 0.01 mg/ml and 0.05 mg/ml. In another alternative, the concentration of the gluconic, gulonic or mannonic acid is in a range of between 0.015 mg/ml and 0.025 mg/ml, such as in an amount of 0.018 mg/ml.

Optionally, a pharmaceutical composition of the invention can further include at least one neutral alcoholic sugar as a stabilizing agent in combination with the aforementioned at least one compound selected from the group consisting of acids derived from neutral alcoholic sugars, lactones of these acids and salts of these acids. Suitable examples of neutral alcoholic sugars include mannitol and sorbitol. Mannitol or sorbitol are excipients which for the preparation of injection drug forms are commercially available in high quality. Although they exhibit lower solubilization power than other alcoholic sugars or similar solubilizers with hydroxyl groups such as maltitol, their advantage consists in the fact that quality requirements and methods of their examination are specified in pharmacopoeias. In compositions according to the invention, neutral alcoholic sugars, such as mannitol and sorbitol, can stabilize the structure of water and thus lower its activity around the reaction center of the oxaliplatinum molecule in the hydrolysis.

Generally, the concentration of a neutral alcoholic sugar, such as mannitol or sorbitol, is sufficiently high enough to stabilize satisfactorily the water structure, and at the same time low enough to reduce the probability of complex formation of platinum with the alcoholic sugar. In a specific embodiment, the concentration is in a range of between 5 mg/ml and 50 mg/ml of the composition. In another specific embodiment, the concentration is in a range of 10 mg/ml and 25 mg/ml of the composition.

Generally, the pharmaceutical compositions of the invention have a pH value in a range of between 3.5 and 7.5. In one specific embodiment, the pH value is in a range of between 3.5 and 6.5. In another specific embodiment, the pH value is in a range of between 3.5 and 5.5. In yet another specific embodiment, the pH value is in a range of between 3.8 and 5.0. In yet another specific embodiment, the pH value is in a range of between 4.0 and 5.0. In yet another specific embodiment, the pH value is in a range of between 3.8 and 4.4. In yet another specific embodiment, the pH value is in a range of between 4.0 and 4.4 (i.e., pH 4.2±0.2). If desired, these embodiments can further include at least one of the aforementioned neutral alcoholic sugars.

In a specific embodiment, a pharmaceutical composition of the invention includes gluconic acid and/or a gluconolactone and/or a salt of gluconic acid as a stabilizing agent in total amount of between 0.01 mg/ml and 0.05 mg/ml (e.g., between 0.01 mg/ml and 0.025 mg/ml), wherein the pH of the composition is in a range of between 3.8 and 5.0. In another specific embodiment, a pharmaceutical composition of the invention includes gluconic acid and/or a gluconolactone and/or a salt of gluconic acid as a stabilizing agent in total amount of between 0.015 mg/ml and 0.025 mg/ml (e.g., 0.018 mg/ml), wherein the pH of the composition is in a range of between 3.8 and 5.0. In another specific embodiment, a pharmaceutical composition of the invention includes gluconic acid and/or a gluconolactone and/or a salt of gluconic acid as a stabilizing agent in total amount of between 0.015 mg/ml and 0.025 mg/ml (e.g., 0.018 mg/ml), wherein the pH of the composition is in a range of between 3.8 and 4.4, more specifically between 4.0 and 4.4 (i.e., pH 4.2±0.2). In one aspect of these specific embodiments, the stabilizing agent includes gluconic acid, a gluconolactone (e.g., delta-gluconolactone) and a salt of gluconic acid. In another aspect of these specific embodiments, the amount of oxaliplatin is in range of 3 mg/ml and 6 mg/ml, such as 5 mg/ml. In yet another aspect of these specific embodiments, the amount of oxaliplatin is 5 mg/ml, and the stabilizing agent includes gluconic acid, a gluconolactone (e.g., delta-gluconolactone) and a salt of gluconic acid.

A suitable example of a pharmaceutically acceptable aqueous solvent that can be employed in the invention includes water. In a specific embodiment, the water has quality for pharmaceutical injections.

The invention also relates to a method of producing the mentioned sterile liquid pharmaceutical composition, characterized in that oxaliplatinum is dissolved in an aqueous solvent, whereupon to this solution of oxaliplatinum is added at least one acid derived from a neutral alcoholic sugar and/or at least one lactone of this acid and/or at least one salt of this acid, and optionally the pH value of the solution is adjusted to a desired pH by addition of an alkali metal hydroxide and/or an alkali earth metal hydroxide, whereupon the obtained solution is sterilized by filtration and filled into individual package units and optionally inertized with nitrogen or argon. When at least one neutral alcoholic sugar is employed, oxaliplatinum is dissolved in a sugar solution maded by dissolving the neutral alcoholic sugar in water to form an oxaliplatinum-sugar solution. Alternatively, the neutral alcoholic sugar is dissolved in an aqueous oxaliplatinum solution, or dissolved in water together with oxaliplatinum.

In one embodiment, a pharmaceutical composition of the invention is prepared by mixing oxaliplatinum and a stabilizing agent that includes a lactone of an acid derived from a neutral alcoholic sugar (e.g., preferably an equilibrium aqueous mixture of a lactone of an acid derived from a neutral alcoholic sugar, and its corresponding acid) to form an oxaliplatinum-stabilizer mixture. The equilibrium aqueous mixture of a lactone and its corresponding acid can be prepared by dissolving the lactone in water, or by any other suitable means (e.g., mixing the lactone and its corresponding acid in water). Specifically, the equilibrium aqueous mixture of a lactone and its corresponding acid can be prepared by dissolving the lactone in water. In a specific embodiment, oxaliplatinum is dissolved in an aqueous solvent to form an aqueous oxaliplatinum solution; and the aqueous oxaliplatinum solution is combined with the stabilizing agent to form the oxaliplatinum-stabilizer mixture. In another specific embodiment, oxaliplatinum is dissolved in an aqueous solution that includes the stabilizing agent to form the oxaliplatinum-stabilizer mixture.

In the embodiments described in the preceding paragraph, if desired, at least one alcoholic sugar (e.g., mannitol and/or sorbitol) can also be mixed with oxaliplatinum and the stabilizing agent. In one example, oxaliplatinum can be dissolved in a sugar solution that includes the alcoholic sugar(s) in a concentration of between 1 mg/ml and 10 mg/ml, specifically between 3 mg/ml and 7 mg/ml (e.g., 5 mg/ml)), to form an oxaliplatinum-sugar solution; and the oxaliplatinum-sugar solution is combined with the stabilizing agent. In another example, the alcoholic sugar(s) (either in solid or as an aqueous sugar solution) is mixed with an aqueous oxaliplatinum solution to form an oxaliplatinum-sugar solution; and the aqueous oxaliplatinum-sugar solution is combined with the stabilizing agent. In yet another example, the alcoholic sugar(s) (either in solid or as an aqueous sugar solution) is mixed with the oxaliplatinum-stabilizer mixture formed by the methods described in the preceding paragraph.

In one specific embodiment, the lactone is derived from gluconic acid and/or gluconic acid and/or mannoic acid. In another specific embodiment, the lactone is a gluconolactone, such as delta-gluconolactone. Typically, the amount of the lactone employed in the invention is in a range of 0.0005 mg/ml-0.5 mg/ml. In one specific embodiment, the amount of the lactone employed in the invention is in a range of 0.01-0.05 mg/ml. In another specific embodiment, the amount of the lactone employed in the invention is in a range of 0.015 mg/ml-0.025 mg/ml (e.g., 0.018 mg/ml).

In the methods of the invention, optionally, the pH of the oxaliplatinum-stabilizer mixture is adjusted to have the desired pH value by addition of an alkali metal hydroxide and/or an alkali earth metal hydroxide to the oxaliplatinum-stabilizer mixture. Typically, the desired pH value is in a range of between about 3.5 and about 7.5. In one example, the desired pH value is in a range of between about 3.5 and about 6.5. In another example, the desired pH value is in a range of between about 3.5 and about 5.5. In yet another example, the desired pH value is in a range of between about 3.8 and about 5.0. In yet another example, the desired pH value is in a range of between about 3.8 and about 4.4. In yet another example, the desired pH value is in a range of between about 4.0 and about 4.4 (i.e., pH 4.2±0.2). When a neutral alcoholic sugar described above is employed in combination with the lactone, the desired pH value typically is in a range of between about 3.5 and about 7.5, alternatively between about 3.5 and about 6.5, alternatively between about 3.5 and about 5.5, alternatively between about 3.8 and about 5.0, alternatively between about 3.8 and about 4.4, or alternatively between about 4.0 and about 4.4 (i.e., pH 4.2±0.2).

In one specific embodiment, a stabilized sterile composition according to the invention is prepared as follows. The active component, oxaliplatinum, is dissolved in water in a concentration of between 3 mg/ml and 6 mg/ml (e.g., 5 mg/ml), optionally under constant stirring, to form a clear aqueous oxaliplatinum solution. Alternatively, the oxaliplatinum is dissolved in a sugar solution, including at least one alcoholic sugar (e.g., mannitol and/or sorbitol), in concentration of between 1 mg/ml and 10 mg/ml, specifically between 3 mg/ml and 7 mg/ml (e.g., 5 mg/ml)), optionally under constant stirring, to form a clear oxaliplatinum-sugar solution. To the oxaliplatinum solution in water or in a sugar solution is further added an aqueous solution of gluconic acid in an amount corresponding to 0.0005 mg/ml-0.5 mg/ml (e.g., 0.01-0.05 mg/ml, 0.015-0.025 mg/ml, or 0.018 mg/ml), and optionally the pH of the resulting solution is adjusted to have the final pH value in a range of between 3.5 and 6.5 (e.g., between 3.5 and 5.5, 3.8 and 5.0, between 4 and 5, between 3.8 and 4.4, or between 4.0 and 4.4 (i.e., pH 4.2±0.2)). The obtained solution is filtered through a filter of porosity 0.22 micrometers and filled into clear colorless vials of glass of 1^{st} hydrolytic class so as the available dose of the active compound in one vial is 50 mg or 100 mg. The vials are sealed with bromobutyl rubber stoppers and secured with an aluminum cap with a polypropylene cover.

In a further specific embodiment, an aqueous solution of the equilibrium mixture of gluconate and gluconic acid, which is prepared by dissolving gluconolactone, is mixed with the oxaliplatinum solution in water or in a sugar solution. The pH value of the resulting solution is then optionally adjusted to 3.5-6.5 (e.g., between 3.5 and 5.5, 3.8 and 5.0, between 4 and 5, between 3.8 and 4.4, or between 4.0 and 4.4 (i.e., pH 4.2±0.2)) by addition of an alkali metal hydroxide and/or an alkali earth metal hydroxide. The amount of the gluconolactone is in a range of 0.0005 mg/ml-0.5 mg/ml. Alternatively, the amount of the gluconolactone is in a range of 0.01 mg/ml-0.05 mg/ml. Alternatively, the amount of the gluconolactone is in a range of 0.015 mg/ml-0.025 mg/ml (e.g., 0.018 mg/ml). Typically, the obtained solution is sterilized by filtration and filled into individual package units and optionally inertized with nitrogen or argon. In one example, the obtained solution is filtered through a filter of porosity 0.22 micrometers and filled into clear colorless vials of glass of 1^{st} hydrolytic class so as the available dose of the active compound in one vial is 50 mg or 100 mg. The vials are sealed with bromobutyl rubber stoppers and secured with an aluminum cap with a polypropylene cover. An example of gluconate that can be used for the preparation of the aqueous solution of the equilibrium mixture of gluconate and gluconic acid is 1% of delta-gluconolactone.

### Exemplification

The examples below explain the composition and method of producing a sterile liquid composition suitable for parenteral administration, containing oxaliplatinum as the active compound, a pharmaceutically acceptable solvent and substances that physically and chemically stabilize the active compound, without limiting in any way the scope of the invention.

### Example A

### Pharmaceutical Compositions Employing Gluconolactone/Gluconic Acid

### Example A1

The active compound oxaliplatinum (5 mg/ml) was dissolved in water for injections. Under constant stirring a clear solution was formed. To this solution was further added an aqueous solution of an equilibrium mixture of gluconolactone and gluconic acid. The equilibrium mixture of gluconolactone and gluconic acid was prepared by dissolving gluconolactone in water (1 g/100 ml). The equilibrium mixture was added to the oxaliplatinum solution until the pH of the resulting mixture was adjusted to pH 3. The obtained solution was filtered through a filter of porosity 0.22 micrometers and filled into clear colorless vials of glass of 1^{st} hydrolytic class. The vials were sealed with bromobutyl rubber stoppers and secured with an aluminum cap covered with polypropylene.

### Example A2

The active compound oxaliplatinum (5 mg/ml) was dissolved in water for injections. Under constant stirring a clear solution was formed. To this solution was further added an aqueous solution of an equilibrium mixture of gluconolactone and gluconic acid. The equilibrium mixture of gluconolactone and gluconic acid was prepared by dissolving gluconolactone in water (1 g/100 ml).). The equilibrium mixture was added to the oxaliplatin solution until the pH of the resulting mixture was a was adjusted to pH 4. The obtained solution was filtered through a filter of porosity 0.22 micrometers and filled into clear colorless vials of glass of 1^{st} hydrolytic class. The vials were sealed with bromobutyl rubber stoppers and secured with an aluminum cap covered with polypropylene.

### Example A3

The active compound oxaliplatinum (5 mg/ml) was dissolved in water for injections. Under constant stirring a clear solution was formed. To this solution was further added an aqueous solution of an equilibrium mixture of gluconolactone and gluconic acid. The equilibrium mixture of gluconolactone and gluconic acid was prepared by dissolving gluconolactone in water (1 g/100 ml). The equilibrium mixture was added to the oxaliplatin solution until the pH of the resulting mixture was adjusted to pH 5. The obtained solution was filtered through a filter of porosity 0.22 micrometers and filled into clear colorless vials of glass of 1^{st} hydrolytic class. The vials were sealed with bromobutyl rubber stoppers and secured with an aluminum cap covered with polypropylene.

### Example A4

The active compound oxaliplatinum (5 mg/ml) was dissolved in water for injections. Under constant stirring a clear solution was formed. To this solution was further added an aqueous solution of an equilibrium mixture of gluconolactone and gluconic acid. The equilibrium mixture of gluconolactone and gluconic acid was prepared by dissolving gluconolactone in water (1 g/100 ml). The equilibrium mixture was added to the oxaliplatin solution until the pH of the resulting mixture was adjusted to pH 6. The obtained solution was filtered through a filter of porosity 0.22 micrometers and filled into clear colorless vials of glass of 1^{st} hydrolytic class. The vials were sealed with bromobutyl rubber stoppers and secured with an aluminum cap covered with polypropylene.

### Example A5 (Reference)

The active compound oxaliplatinum (5 mg/ml) was dissolved in water for injections. Constant stirring affords a clear solution which was filtered through a filter of porosity 0.22 micrometers and was filled into clear colorless vials of glass of 1^{st} hydrolytic class. The vials were sealed with bromobutyl rubber stoppers and secured with an aluminum cap covered with polypropylene.

### Example A6 (Stability Study)

The vials prepared according to Examples A1-A5 were subjected to accelerated stability study in the “stopper up” position at 40° C. and 75% relative humidity. At the beginning (T₀) and after 6 months (6M) the samples were assayed for content of the active compound and purity. The analyses were performed by liquid chromatography. The results are given in Table I.

TABLE 1
Results of analyses at the beginning of the study and after 6 months

| | Content of | | Content of | | | |
|---|---|---|---|---|---|---|
| | active | | oxalic | | Sum of | |
| | compound | | acid | | impurities | |
| | (mg/ml) | | (% wt) | | (% wt) | |
| Example | T₀ | 6 M | T₀ | 6 M | T₀ | 6 M |
| Example A1 | 4.87 | 5.03 | 0.53 | 0.34 | 0.55 | 1.23 |
| Example A2 | 5.04 | 4.96 | 0.15 | 0.12 | 0.18 | 0.28 |
| Example A3 | 5.04 | 5.07 | 0.10 | 0.08 | 0.12 | 0.15 |
| Example A4 | 4.97 | 5.02 | 0.44 | 0.64 | 0.46 | 0.78 |
| Example A5 | 4.94 | 4.89 | 0.17 | 0.17 | 0.19 | 0.33 |
| (reference) | | | | | | |

The data in Table 1 shows the positive effect of addition of aqueous solution of an equilibrium mixture of gluconolactone and gluconic acid on the stability of oxaliplatinum solution in the range of pH 4-5. In comparison with a simple solution of oxaliplatinum in water (reference example A5), after storing for 6 months at 40° C. and 75% relative humidity, the compositions according to Examples A2 and A3 contain a significantly lower amount of oxalic acid as well as lower total amount of impurities.

### Example B

### Pharmaceutical Compositions Employing Mannitol and Gluconolactone/Gluconic Acid

### Example B1 (Reference)

Mannitol (25 mg/ml) and the active compound oxaliplatinum (5 mg/ml) were dissolved in water for injections. Under constant stirring a clear solution was formed. The obtained solution was filtered through a filter of porosity 0.22 micrometers and filled into clear colorless vials of glass of 1^{st} hydrolytic class. The vials were sealed with bromobutyl rubber stoppers and secured with an aluminum cap covered with polypropylene.

### Example B2 (Reference)

Mannitol (12.5 mg/ml) and the active compound oxaliplatinum (5 mg/ml) were dissolved in water for injections. Under constant stirring a clear solution was formed. The obtained solution was filtered through a filter of porosity 0.22 micrometers and filled into clear colorless vials of glass of 1^{st} hydrolytic class. The vials were sealed with bromobutyl rubber stoppers and secured with an aluminum cap covered with polypropylene.

### Example B3

Mannitol (25 mg/ml) and the active compound oxaliplatinum (5 mg/ml) were dissolved in water for injections. Under constant stirring a clear solution was formed to which was further added an aqueous solution of the equilibrium mixture of gluconolactone and gluconic acid. The equilibrium mixture of gluconolactone and gluconic acid was prepared by dissolving gluconolactone in water (1 g/100 ml). The equilibrium mixture was added to the oxaliplatin solution until pH of the resulting mixture was adjusted to pH 4-5. The obtained solution was filtered through a filter of porosity 0.22 micrometers and filled into clear colorless vials of glass of 1^{st} hydrolytic class. The vials were sealed with bromobutyl rubber stoppers and secured with an aluminum cap covered with polypropylene.

### Example B4

Mannitol (12.5 mg/ml) and the active compound oxaliplatinum (5 mg/ml) were dissolved in water for injections. Under constant stirring a clear solution was formed to which was further added aqueous solution of an equilibrium mixture of gluconolactone and gluconic acid. The equilibrium mixture of gluconolactone and gluconic acid was prepared by dissolving gluconolactone in water (1 g/100 ml). The equilibrium mixture was added to the oxaliplatin solution until the pH of the resulting mixture was adjusted to 4-5. The obtained solution was The obtained solution was filtered through a filter of porosity 0.22 micrometers and filled into clear colorless vials of glass of 1^{st} hydrolytic class. The vials were sealed with bromobutyl rubber stoppers and secured with an aluminum cap covered with polypropylene.

### Example B5 (Reference)

The active compound oxaliplatinum (5 mg/ml) was dissolved in water for injections. Constant stirring affords a clear solution which was filtered through a filter of porosity 0.22 micrometers and filled into clear colorless vials of glass of 1^{st} hydrolytic class. The vials were sealed with bromobutyl rubber stoppers and secured with an aluminum cap covered with polypropylene.

### Example B6 (Stability Study)

The vials prepared according to Examples B1-B5 were subjected to accelerated stability study in the “stopper up” position at 40° C. and 75% relative humidity. At the beginning and after 3 months the samples were assayed for the content of the active compound and purity. The analyses were performed by liquid chromatography. The results are given in Table 2.

TABLE 2
Results of analyses at the beginning of the study and after 3 months

| | Content | | Content of | | | |
|---|---|---|---|---|---|---|
| | of active | | oxalic | | Sum of | |
| | compound | | acid | | impurities | |
| | (% wt) | | (% wt) | | (% wt) | |
| Example | T₀ | 3 M | T₀ | 3 M | T₀ | 3 M |
| Example B1 | 100 | 98.6 | 0.11 | 0.14 | 0.12 | 0.45 |
| Example B2 | 100 | 97.7 | 0.11 | 0.13 | 0.12 | 0.36 |
| Example B3 | 100 | 98.8 | 0.11 | 0.15 | 0.12 | 0.32 |
| Example B4 | 100 | 98.4 | 0.11 | 0.15 | 0.12 | 0.21 |
| Example B5 | 100 | 97.4 | 0.17 | 0.19 | 0.19 | 0.32 |

The data in Table 2 shows the fact that after storing for 3 months at elevated temperature and high relative humidity, the compositions prepared according to Examples B1 and B2 contain lower concentrations of the hydrolysis product—oxalic acid—compared with a simple solution of oxaliplatinum in water for injections (reference Example B5).

Further, compositions prepared according to Examples B3 and B4, i.e. with addition of solution of an equilibrium mixture of gluconolactone and gluconic acid, after 3 months' storing at elevated temperature and relative humidity exhibit higher purity (expressed as the sum of impurities), even as compared with Examples B1 and B2. This unequivocally shows not only the advantage of using an alcoholic sugar as stabilizer but a still greater advantage of using a mixture of alcoholic sugar and an equilibrium mixture of gluconolactone and gluconic acid.

1. A pharmaceutical composition, comprising oxaliplatinum, a pharmaceutically acceptable aqueous solvent and a stabilizing agent in a stabilizingly effective amount, wherein the stabilizing agent includes at least one compound selected from the group consisting of acids derived from neutral alcoholic sugars, lactones of these acids and salts of these acids.

2. The pharmaceutical composition of claim 1, wherein the stabilizing agent includes acids derived from mannitol and/or sorbitol and/or lactones of these acids and/or salts of these acids.

3. The pharmaceutical composition of claim 1, wherein the stabilizing agent includes an acid derived from neutral alcoholic sugars which in aqueous solution is at equilibrium with its lactone.

4. The pharmaceutical composition of claim 1, wherein the stabilizing agent includes gluconic acid and/or gulonic acid and/or mannonic acid and/or lactones of these acids and/or salts of these acids.

5. The pharmaceutical composition of claim 1, wherein the salts of acids derived from neutral alcoholic sugars are alkali metal salts and/or alkali earth metal salts of these acids.

6. The pharmaceutical composition of claim 1, wherein the salts of acids derived from neutral alcoholic sugars are sodium and/or potassium and/or magnesium and/or calcium salts of these acids.

7. The pharmaceutical composition of claim 1, wherein the pharmaceutically acceptable aqueous solvent is water.

8. The pharmaceutical composition of claim 7, the water is in the quality water for injections.

9. The pharmaceutical composition of claim 1, wherein the stabilizing agent includes at least one acid derived from neutral sugars and/or at least one lactone of this acid and/or at least one salt of this acid, in total amount of between 0.0005 mg/ml and 0.5 mg/ml of the composition.

10. The pharmaceutical composition of claim 1, wherein oxaliplatinum is in an amount of between 3 mg/ml and 6 mg/ml of the composition.

11. The pharmaceutical composition of claim 1, wherein its pH value is between 3.5 and 6.5.

12. The pharmaceutical composition of claim 11, wherein its pH value is between 4 and 5.

13. A method of producing a pharmaceutical composition, comprising: dissolving oxaliplatinum in an aqueous solvent to form an aqueous oxaliplatinum solution; combining the aqueous oxaliplatinum solution with a stabilizing agent that includes at least one compound selected from the group consisting of acids derived from neutral alcoholic sugars, lactones of these acids and salts of these acids to form an oxaliplatinum-stabilizer mixture; optionally adjusting a pH value of the oxaliplatinum-stabilizer mixture to pH 3.5-6.5 by addition of an alkali metal hydroxide and/or an alkali earth metal hydroxide to the oxaliplatinum-stabilizer mixture to thereby form a stable oxaliplatinum solution; sterilizing the stable oxaliplatinum solution; and filling into individual package units and optionally inertized with nitrogen or argon.

14. The pharmaceutical composition of claim 1, wherein the stabilizing agent includes at least one compound selected from the group consisting of monocarboxylic acids derived from neutral alcoholic sugars, lactones of these acids and salts of these acids.

15. The pharmaceutical composition of claim 1, wherein the stabilizing agent includes at least one compound selected from the group consisting of monocarboxylic acids derived from mannitol or sorbitol, lactones of these acids, and salts of these acids.

16. The pharmaceutical composition of claim 1, wherein the stabilizing agent includes at least one monocarboxylic acid derived from a neutral alcoholic sugar, which in aqueous solution is at equilibrium with its lactone.

17. The pharmaceutical composition of claim 1, wherein the stabilizing agent includes at least one acid derived from neutral sugars and/or at least one lactone of this acid and/or at least one salt of this acid, in total amount of 0.005 mg/ml and 0.1 mg/ml of the composition.

18. The pharmaceutical composition of claim 17, wherein the stabilizing agent includes at least one acid derived from neutral sugars and/or at least one lactone of this acid and/or at least one salt of this acid, in total amount of 0.01 mg/ml and 0.05 mg/ml of the composition.

19. The pharmaceutical composition of claim 18, wherein the stabilizing agent includes at least one acid derived from neutral sugars and/or at least one lactone of this acid and/or at least one salt of this acid, in total amount of 0.015 mg/ml and 0.025 mg/ml of the composition.

20. The pharmaceutical composition of claim 19, wherein the stabilizing agent includes gluconic acid and/or a gluconolactone and/or a salt of gluconic acid.

21. The pharmaceutical composition of claim 19, wherein oxaliplatinum is present in an amount of between 3 mg/ml and 6 mg/ml of the composition.

22. The pharmaceutical composition of claim 21, wherein the composition has a pH value in a range of between 3.8 and 5.0

23. The pharmaceutical composition of claim 22, wherein oxaliplatinum is present in an amount of 5 mg/ml of the composition.

24. The pharmaceutical composition of claim 23, wherein the composition has a pH value in a range of between 3.8 and 4.4.

25. A method of producing a pharmaceutical composition, comprising: mixing oxaliplatinum and a stabilizing agent that includes a lactone of an acid derived from a neutral alcoholic sugar to form an oxaliplatinum-stabilizer mixture; and optionally adjusting a pH value of the oxaliplatinum-lactone mixture to a pH between 3.5 and 6.5 by addition of an alkali metal hydroxide and/or an alkali earth metal hydroxide to the oxaliplatinum-lactone mixture.

26. The method of claim 25, wherein the lactone of the stabilizing agent is in equilibrium with its corresponding acid in water.

27. The method of claim 26, wherein the oxaliplatinum and the stabilizing agent is mixed by:
dissolving oxaliplatinum in an aqueous solvent to form an aqueous oxaliplatinum solution; and
combining the aqueous oxaliplatinum solution with the stabilizing agent to form the oxaliplatinum-stabilizer mixture.

28. The method of claim 26, wherein the lactone is derived from gluconic acid and/or gulonic acid and/or mannonic acid.

29. The method of claim 28, wherein the lactone is a gluconolactone.

30. The method of claim 29, wherein the lactone is delta-gluconolactone.
